(19) United States
(12) Patent Application Publication
(10) Pub. No.: US 2021/0079436 A1
(43) Pub. Date: Mar. 18, 2021

(54) METHOD FOR PREPARING RHAMNOLIPIDS

(71) Applicant: Hong Kong Baptist University, Hong Kong (CN)

(72) Inventors: Guneet Kaur, New Delhi (IN); Woon Chung Jonathan Wong, Hong Kong (CN)

(21) Appl. No.: 16/843,976

(22) Filed: Apr. 9, 2020

Related U.S. Application Data
(60) Provisional application No. 62/899,797, filed on Sep. 13, 2019.

(51) Int. Cl. C12P 19/44

(52) U.S. Cl. CPC C12P 19/44

(57) ABSTRACT

The present disclosure relates to a method for producing rhamnolipids.

### CROSS REFERENCE OF RELATED APPLICATION

This application claims the benefit of priority of U.S. Provisional Patent Application Ser. No. 62/899,797 filed on Sep. 13, 2019, which is hereby incorporated by reference in its entirety for all purposes.

### TECHNICAL FIELD

The present disclosure generally relates to a method for preparing rhamnolipids. More particularly, the present disclosure relates to a method for preparing rhamnolipids using anaerobic digestate.

### BACKGROUND

Surfactants are amphiphilic, surface active molecules and an important class of chemicals with myriad applications in household, agriculture, pharmaceuticals, food, cosmetics and health. The conventional sourcing of surfactants has been made by the chemical/synthetic feedstocks. Their ecotoxicity, bioaccumulation and biodegradability has raised serious concerns in the past which led to research initiatives towards the production of biosurfactants by growing microorganisms such as bacteria, yeast, filamentous fungi on different carbon sources. Biosurfactants offer superior environmental compatibility and excellent functional properties. These are usually produced during stationary phases of microbial growth as secondary metabolites. The major classes of biosurfactants include glycolipids, lipoproteins or lipopeptides, phosphoproteins, fatty acids and phospholipids, and polymeric surfactants.

Among these, the greatest interest has focused on glycolipids and particularly rhamnolipids due to their outstanding applications making it the most popular biosurfactant on the global market. Rhamnolipids are glycosides consisting of one (mono-rhamnolipid) or two rhamnose-units (di-rhamnolipid) as the glycon portion and one to three β-hydroxyfatty acid units as the aglycon portion. In addition to their excellent emulsification, wetting and foaming properties and applications thereof, rhamnolipids also exhibit some inimitable applications, such as bioremediation and enhanced oil recovery, antimicrobial properties, and cosmetic uses.

High cost, limited availability and complexity of feedstock are major challenges to rhamnolipids production. Substrates cost around 50% of the total production cost. Hence, the use of renewable and waste resources such as lignocellulosic biomass, e.g., wheat straw, empty fruit bunch, industrial residues, such as soapstock, glycerol, cheese whey, residual oils, and waste frying oils have been explored to produce next generation biosurfactants, including rhamnolipids. While the motive is to develop more favourable techno-economic processes for reducing the production costs, there are at least two major drawbacks in this approach.

First, substrates such as lignocellulosic hydrolysates e.g. wheat straw, rice straw, rice bran etc. are recalcitrant substances and require complicated, and time and cost-intensive pre-treatment steps such as thermal treatment, acid hydrolysis, enzyme hydrolysis to yield simple sugars which are subsequently utilized as feedstock for rhamnolipid production. Furthermore, hydrolysates, especially lignocellulosics, usually contain toxic and harsh substances, such as furfural and 5-hydroxymethyfurfural, which can have negative effects on biomass growth and metabolite production. Additional separation steps involving the usage of harsh chemicals are required to remove these inhibitors before hydrolysate can be used as substrate for bio-production.

Even while using waste feedstock, the growth and production medium is usually supplemented with expensive and refined chemicals in the form of mineral salts, growth factors, vitamins, nitrogen sources, etc. In fact, waste feedstock is used as a carbon source at a small concentration in the total medium while majority of the medium is made up of refined substrates. Thus, the use of a waste substrate as ‘complete’ medium for rhamnolipids production is not known.

There is therefore a need for an improved method for producing rhamnolipids that addresses or overcomes at least some of the aforementioned issues.

### SUMMARY

Accordingly, it is an objective of the present disclosure to provide a bio-based, cost-effective and sustainable method for producting rhamnolipids.

In a first aspect, provided a method for producing rhamnolipids, the method comprising: providing a culture medium comprising an anaerobic digestate and a host cell which produces rhamnolipids; cultivating the host cell under conditions that the host cell produces the rhamnolipids; recovering the rhamnolipids; and optionally isolating the rhamnolipids.

In a first embodiment of the first aspect, provided herein is the method of the first aspect, wherein the anaerobic digestate is untreated.

In a second embodiment of the first aspect, provided herein is the method of the first aspect, wherein the anaerobic digestate is substantially the only carbon source and nitrogen source in the culture medium.

In a third embodiment of the first aspect, provided herein is the method of the first aspect, wherein the host cell is Acinetobacter calcoaceticus.

In a fourth embodiment of the first aspect, provided herein is the method of the first aspect, wherein the culture medium has a carbohydrates concentration of 10 to 30 g/L and a carbon to nitrogen ratio mass ratio between 7 to 22.

In a fifth embodiment of the first aspect, provided herein is the method of the first aspect, wherein the host cell is cultivated at a temperature of 20° C. to 60° C.

In a sixth embodiment of the first aspect, provided herein is the method of the first aspect, wherein the culture medium has a pH of 6 to 9.

In a seventh embodiment of the first aspect, provided herein is the method of the first aspect, further comprising adding one or more additional portions of a feed anaerobic digestate into the culture medium during about the mid-exponential growth phase of the host cell to about the late-exponential growth phase of the host cell thereby forming a fermentation culture having a carbohydrate concentration in the fermentation culture between 10 to 30 g/L and a carbon to nitrogen ratio mass ratio of 7 to 22.

In a second aspect, provided herein is a method for producing rhamnolipids, the method comprising: providing a culture medium comprising an anaerobic digestate and Acinetobacter calcoaceticus under conditions that the Acinetobacter calcoaceticus produces the rhamnolipids, wherein the conditions comprise cultivating the Acinetobacter calcoaceticus until about mid-exponential growth phase of the Acinetobacter calcoaceticus to the late-exponential growth phase of the Acinetobacter calcoaceticus; adding one or more additional portions of a feed anaerobic digestate into the culture medium thereby forming a fermentation culture having a carbohydrate concentration in the fermentation culture between 10 to 30 g/L and a carbon to nitrogen ratio mass ratio of 7 to 22; recovering the rhamnolipids; and optionally isolating the rhamnolipids, wherein the anaerobic digestate and the one or more portions of the feed anaerobic digestate are substantially the only carbon source and nitrogen source in the culture medium.

In a first embodiment of the second aspect, provided herein is the method of the second aspect, wherein the anaerobic digestate and the one or more portions of the feed anaerobic digestate are untreated.

In a second embodiment of the second aspect, provided herein is the method of the first embodiment of the second aspect, wherein the anaerobic digestate and the one or more portions of the feed anaerobic digestate are prepared by anaerobic digestion of food waste.

In a third embodiment of the second aspect, provided herein is the method of the second embodiment of the second aspect, wherein the anaerobic digestate and the one or more portions of the feed anaerobic digestate have a pH between 6 to 9 an electrical conductivity between 7.5 to 16 mS/cm.

In a fourth embodiment of the second aspect, provided herein is the method of the second aspect, wherein the culture medium has a pH between 6 and 9 and a temperature of 20° C. to 60° C.

In a fifth embodiment of the second aspect, provided herein is the method of the second aspect, further comprising: providing a first pre-culture medium comprising Acinetobacter calcoaceticus; combining the first pre-culture medium and a first portion of the anaerobic digestate thereby forming a second pre-culture medium; and combining the second pre-culture medium with a second portion of the anaerobic digestate thereby forming the culture medium.

In a sixth embodiment of the second aspect, provided herein is the method of the second aspect, further comprising: cultivating the fermentation culture between 4 to 50 hours (h) before the step of recovering the rhamnolipids.

In a seventh embodiment of the second aspect, provided herein is the method of the second aspect, wherein the method comprises: providing a culture medium comprising an anaerobic digestate and Acinetobacter calcoaceticus under conditions that the Acinetobacter calcoaceticus produces the rhamnolipids, wherein the conditions comprise cultivating the Acinetobacter calcoaceticus until about late-exponential growth phase of the Acinetobacter calcoaceticus; adding one or more additional portions of a feed anaerobic digestate into the culture medium thereby forming a fermentation culture having a carbohydrate concentration in the fermentation culture between 10 to 30 g/L and a carbon to nitrogen ratio mass ratio between 10 to 22; cultivating the fermentation culture between 20 to 30 h, wherein the fermentation culture has a pH between 7.5 to 9 and a temperature between 50° C. and 60° C.; harvesting the rhamnolipids; and optionally isolating the rhamnolipids, wherein the anaerobic digestate and the one or more portions of the feed anaerobic digestate are substantially the only carbon source and nitrogen source in the culture medium and the fermentation culture, and wherein the anaerobic digestate and the one or more portions of the feed anaerobic digestate are untreated; have a pH between 7.5 to 9; and an electrical conductivity between 7.5 to 16 mS/cm.

In an eighth embodiment of the second aspect, provided herein is the method of the seventh embodiment of the second aspect, further comprising providing a first pre-culture medium comprising Acinetobacter calcoaceticus; combining the first pre-culture medium and a first portion of the anaerobic digestate thereby forming a second pre-culture medium; and combining the second pre-culture medium with a second portion of the anaerobic digestate thereby forming a culture medium comprising anaerobic digestate and Acinetobacter calcoaceticus.

In a ninth embodiment of the second aspect, provided herein is the method of the seventh embodiment of the second aspect, wherein the anaerobic digestate and the one or more portions of the feed anaerobic digestate are prepared by anaerobic digestion of food waste.

In a tenth embodiment of the second aspect, provided herein is the method of the seventh embodiment of the second aspect, wherein the fermentation culture produces rhamnolipids at a concentration of 8 to 12 g/L.

In an eleventh embodiment of the second aspect, provided herein is the method of the seventh embodiment of the second aspect, wherein the rhamnolipids are isolated by liquid-liquid extraction of the fermentation culture with an organic solvent.

Those skilled in the art will appreciate that the invention described herein is susceptible to variations and modifications other than those specifically described.

The present invention includes all such variation and modifications. The invention also includes all the steps and features referred to or indicated in the specification, individually or collectively, and any and all combination or any two or more of the steps or features.

Other aspects and advantages of the present invention will be apparent to those skilled in the art from a review of the ensuing description.

### BRIEF DESCRIPTION OF THE DRAWINGS

The above and other objects and features of the present invention will become apparent from the following description of the invention, when taken in conjunction with the accompanying drawings, in which:

FIG. 1 shows an exemplary process flow diagram for rhamnolipids production from anaerobic digestate in accordance with certain embodiments described herein.

FIG. 2 shows the effect of carbon/nitrogen mass ratio (C/N) of anaerobic digestate on rhamnolipids production in accordance with certain embodiments described herein. Values are indicated as relative to the best condition found from the range in all experiments (i.e., C/N 22).

FIG. 3 shows the effect of anaerobic digestate pH on culture growth and rhamnolipids production in accordance with certain embodiments described herein. Values are indicated as relative to the best condition found from the range in all experiments (i.e., pH 7.5).

FIG. 4 shows the effect of cultivation time on culture growth and rhamnolipids production in accordance with certain embodiments described herein. Values are indicated as relative to the best condition found from the range in all experiments (i.e., 48 h).

FIG. 5 shows biomass growth kinetics during fed-batch fermentation on anaerobic digestate used as both batch medium and feed by A. calcoaceticus in accordance with certain embodiments described herein. Feeding of the anaerobic digestate in pulse addition mode is done at 20 h in accordance with certain embodiments described herein. Biomass growth is expressed as colony forming units (CFU/mL).

FIG. 6 shows rhamnolipids production in shake flask and in fed-batch fermentation in bioreactor in accordance with certain embodiments described herein.

FIG. 7A shows Fourier-transform infrared spectroscopy (FTIR) spectra of rhamnolipids obtained from bioreactor fermentation. The characteristic bands of rhamnolipids and their respective positions are indicated for (i) rhamnolipids standard, (ii) anaerobic digestate-derived rhamnolipids prepared in accordance with certain embodiments described herein, and (iii) synthetic medium-derived rhamnolipids.

FIG. 7B shows characteristics bands in anaerobic digestate-derived rhamnolipids prepared in accordance with certain embodiments described herein.

FIG. 8A shows tandem mass spectrometry (MS-MS) spectra of the individual peaks shown in MS spectra for extracted rhamnolipid sample from bioreactor prepared in accordance with certain embodiments described herein. Peak at m/z 476 shows the respective congener of rhamnolipids present in the sample. Congener is indicated on the side of corresponding peak in the scan.

FIG. 8B shows MS-MS spectra of the individual peaks shown in MS spectra for extracted rhamnolipid sample from bioreactor prepared in accordance with certain embodiments described herein. Peak at m/z 503 shows the respective congener of rhamnolipids present in the sample. Congener is indicated on the side of corresponding peak in the scan.

FIG. 8C shows MS-MS spectra of the individual peaks shown in MS spectra for extracted rhamnolipid sample from bioreactor prepared in accordance with certain embodiments described herein. Peak at m/z 529 shows the respective congener of rhamnolipids present in the sample. Congener is indicated on the side of corresponding peak in the scan.

FIG. 8D shows MS-MS spectra of the individual peaks shown in MS spectra for extracted rhamnolipid sample from bioreactor prepared in accordance with certain embodiments described herein. Peak at m/z 531 shows the respective congener of rhamnolipids present in the sample. Congener is indicated on the side of corresponding peak in the scan.

FIG. 8E shows MS-MS spectra of the individual peaks shown in MS spectra for extracted rhamnolipid sample from bioreactor prepared in accordance with certain embodiments described herein. Peak at m/z 621 shows the respective congener of rhamnolipids present in the sample. Congener is indicated on the side of corresponding peak in the scan.

FIG. 8F shows MS-MS spectra of the individual peaks shown in MS spectra for extracted rhamnolipid sample from bioreactor prepared in accordance with certain embodiments described herein. Peak at m/z 649 shows the respective congener of rhamnolipids present in the sample. Congener is indicated on the side of corresponding peak in the scan.

FIG. 8G shows MS-MS spectra of the individual peaks shown in MS spectra for extracted rhamnolipid sample from bioreactor prepared in accordance with certain embodiments described herein. Peak at m/z 676 shows the respective congener of rhamnolipids present in the sample. Congener is indicated on the side of corresponding peak in the scan.

FIG. 8H shows MS-MS spectra of the individual peaks shown in MS spectra for extracted rhamnolipid sample from bioreactor prepared in accordance with certain embodiments described herein. Peak at m/z 677 shows the respective congener of rhamnolipids present in the sample. Congener is indicated on the side of corresponding peak in the scan.

FIG. 9 shows surfactant properties of anaerobic digestate-derived rhamnolipids prepared in accordance with certain embodiments described herein. Emulsification capacity of rhamnolipids is compared with synthetic surfactant Tween 80.

### DETAILED DESCRIPTION

Provided herein is a cost-effective and efficient method for producing rhamnolipids from readily available economic raw materials.

Throughout the present specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the present invention.

Furthermore, throughout the present specification and claims, unless the context requires otherwise, the word “include” or variations such as “includes” or “including”, will be understood to imply the inclusion of a stated integer or group of integers but not the exclusion of any other integer or group of integers.

Other definitions for selected terms used herein may be found within the detailed description of the present invention and apply throughout. Unless otherwise defined, all other technical terms used herein have the same meaning as commonly understood to one of ordinary skill in the art to which the invention belongs.

Provided herein is a method for producing rhamnolipids, the method comprising: providing a culture medium comprising an anaerobic digestate and a host cell which produces rhamolipids; cultivating the host cell under conditions that the host cell produces the rhamnolipids; recovering the rhamnolipids; and optionally isolating the rhamnolipids. Allowing the host cell to produce the rhamnolipids may include allowing the host cell to secrete the rhamnolipids.

The rhamnolipids produced by the methods described herein can be any one or more rhamnolipids selected from the group consisting mono-rhamnolipids and di-rhamnolipids. Exemplary rhamnolipids, include but are not limited to Rha-C10-C10; Rha-Rha-C10-C10; Rha-C10-C8; Rha-C10-C12:1; Rha-Rha-C10-C8; Rha-Rha-C10-C12; Rha-Rha-C10-C12:1; and Rha-C10-C12. In certain embodiments, the rhamnolipids produced by the methods described herein are one or more of the rhamnolipids described in Table 1.

The host cell may for example be selected from a bacterial isolate that has been found to produce rhamnolipids, such as Acinetobacter calcoaceticus, Renibacterium salmoninarum, Cellulononas cellulans, Nocardioides sp., Tetragenococcus koreensis, Burkholderia glumae, Burkholderia pseudomallei, Burkholderia plantarii, Burkholderia thailandensis, Myxococcus sp., Enterobacter asburiae, Enterobacter hormaechei, Pantoea stewartii, Pseudomonas alcaligenes, Pseudomonas aeruginosa, Pseudomonas cepacia, Pseudomonas sp. EP-3, Pseudomonas chlororaphis, Pseudomonas clemancea, P. collierea, P. fluorescens, P. putida, P. luteola, P. stutzeri or P. teessidea. In certain embodiments, more than one host cell can be used in the methods described herein. For example, 2, 3, 4, 6, 7, 8, 9, or 10 different host cells can be used in the methods described herein.

In certain embodiments, the Acinetobacter calcoaceticus is selected from Acinetobacter calcoaceticus NRRL B-59190, Acinetobacter calcoaceticus NRRL B-59191, Acinetobacter junii BD and Acinetobacter calcoaceticus BU-03.

The problems facing next-generation rhamnolipid production demand the search for more appropriate feedstocks and production methods for a sustainable technology with improved technical, economical and marketable aspects.

In this regard, the anaerobic digestate that is the leftover material remaining after anaerobic digestion (AD) process can be a suitable feedstock for the methods described herein. AD involves decomposition of biodegradable materials by the action of microorganisms in the absence of oxygen to produce methane rich biogas as renewable energy and nutrient rich anaerobic digestate. In the recent few years, AD has been largely promoted to treat the increasing amounts of food waste, which is being generated globally.

Since anaerobic digestate is rich in nutrients, it could be used as a potential feedstock for production of rhamnolipids. The use of anaerobic digestate for products, such as proteinaceous biomass by cultivating algae, biopesticides from industrial wastewater and secondary sludge from wastewater treatment plant, and construction material from dried manure fibers of AD manure has been explored. However, anaerobic digestate has never been used for rhamnolipids production.

The anaerobic digestate can be anaerobically digested organic material, such as organic waste. The organic waste can be selected from food waste, organic byproducts of manufacturing processes, fats, oils, lipids, grease, yard waste, manure, biosolids, digestible organic materials, and any combination thereof. In certain embodiments, the anaerobic digestate is derived from anaerobically digested food waste.

The reports in literature using waste streams for rhamnolipids production invariably use pure/refined nutrients as co-substrates in the production medium along with waste substrate but do not use the waste as the whole or complete medium. Benincasa et al. (2004) used soapstock at only 2% (w/v) along with mineral salts medium, yeast extract and sodium nitrate as nitrogen source to produce 13.8 g/L rhamnolipids using P. aeruginosa LBI strain in 86 h. Nitschke et al. (2005) also produced rhamnolipids using mineral salts medium and 2% (w/v) soybean soapstock waste as carbon source. With the addition of refined minerals, nitrogen source and trace elements, they produced 11.7 g/L rhamnolipids using the same strain. Gudina et al. (2015) used molasses and cornsteep liquor to produce 3.2 g/L rhamnolipids in 72 h using P. aeruginosa #112. The medium was supplemented with additional nutrients and salts provided by commercial LB medium. Dong et al. (2016) used soybean oil supplemented with sodium nitrate and mineral salts to produce 4 g/L rhamnolipids concentration using Acinetobacter junii in 120 h. Recently, Perez-Armendariz et al. (2019) used 3% (v/v) canola oil along with mineral salts to produce a rhamnolipids concentration of 3.6 g/L using P. aeruginosa.

The above literature reports clearly show that the waste source is usually not used as the whole medium or complete source of nutrients for rhamnolipids production and expensive, refined chemicals are invariably added to support biomass growth and rhamnolipids production. In the present invention, digestate is the complete medium which allows both biomass and rhamnolipids production. With no addition of expensive nutrients and following the inventors' developed fed-batch fermentation method, a high biomass concentration is produced which is capable of producing rhamnolipids at a multi-gram per litre scale in a production period of, e.g., 42 h, which is much shorter than conventional methods.

Advantageously, the anaerobic digestate can be used untreated in the methods described herein. Untreated anaerobic digestate refers to anaerobic digestate that has not been subjected to pre-treatment steps (other than, e.g., adjusting the pH of the anaerobic digestate) prior to use in the methods described herein. Such pre-treatment steps can include thermal treatment, acid hydrolysis, enzyme hydrolysis, or purification to remove toxic and/or inhibitory substances. In certain embodiments, the anaerobic digestate is the culture medium.

The growth of the host cell can be categorized in four different growth phases: the lag phase, the exponential growth phase, the stationary phase, and the death phase.

During the lag phase, the host cells adapt themselves to the growth conditions. During this phase, the individual host cells are maturing and not yet able to divide. During the lag phase of the host cell growth cycle, synthesis of RNA, enzymes and other molecules occurs. The exponential growth phase (sometimes called the logarithmic phase or the log phase) is a period characterized by host cell doubling. The number of new host cells appearing per unit time is proportional to the present population. If growth is not limited, doubling will continue at a constant rate so both the number of cells and the rate of population increase doubles with each consecutive time period. For this type of exponential growth, plotting the natural logarithm of the host cell number against time produces a straight line. The slope of this line is the specific growth rate of the host cell, which is a measure of the number of divisions per cell per unit time. The actual rate of this growth depends upon the growth conditions, which affect the frequency of host cell division events and the probability of both daughter cells surviving. Exponential growth cannot continue indefinitely, however, because the culture medium is soon depleted of nutrients and filled with secreted metabolites and/or toxic molecules.

The mid-exponential phase refers to about the midpoint of the total time of exponential phase. Reference to the late exponential phase refers to the second half of the total exponential phase.

The stationary phase is often due to a growth-limiting factor, such as the depletion of an essential nutrient, and/or the formation of an inhibitory product. Stationary phase results from a situation in which growth rate and death rate are equal. The number of new cells created is limited by the growth factor and as a result the rate of cell growth matches the rate of cell death.

During the death phase, the host cells die. This could be due to lack of nutrients, cell overcrowding, a temperature which is too high or low, or other change in condtitions.

The growth phase of the cell culture can be monitored using any number of conventional methods, such as monitoring at least one of the optical density (typically at 600 nm), disolved oxygen, disolved carbon dioxide, pH, and/or sugars present in the cell culture as a function of time.

Cultivating the host cell under conditions that the host cell produces the rhamnolipids may comprise cultivating the host cell in at least one of the exponential growth phase, the stationary phase, and the death phase. In certain embodiments, cultivating the host cell under conditions that the host cell produces the rhamnolipids may comprise cultivating the host cell in the mid-exponential phase to late-exponential phase and/or stationary phase.

Cultivation time required to reach the mid-exponential phase to the late exponential phase can depend on a number of factors, but generally ranges between 10 to 30 h. In certain embodiments, the cultivation time required to reach the mid-exponential phase to the late exponential phase ranges between 12 to 28; 14 to 26; 16 to 24; or 18 to 22 h. In certain embodiments, the cultivation time required to reach the late exponential phase ranges between 12 to 28; 14 to 26; 16 to 24; or 18 to 22 h. In certain embodiments, the cultivation time required to reach the late exponential phase is about 20 h.

The concentration of carbohydrates in the culture medium can be 1 to 50 g/L. In certain embodiments, the concentration of carbohydrates in the culture medium is 5 to 50 g/L; 5 to 45 g/L; 5 to 40 g/L; 5 to 35 g/L; 10 to 30 g/L; 5 to 30 g/L; 5 to 25 g/L; 5 to 20 g/L; 5 to 15 g/L; or 5 to 10 g/L. In certain embodiments, the concentration of carbohydrates may vary depending on the growth phase of the host cell. In certain embodiments, the cell culture in the lag phase may have a carbohydrate concentration between 20 to 50 g/L; 20 to 45 g/L; 20 to 40 g/L; 20 to 35 g/L; or 25 to 35 g/L.

As the cell culture enters the exponential growth phase carbohydrates present in the cell culture are metabolized by the host cell, which decreases the carbohydrate concentration in the cell culture. In order to avoid limiting host cell growth, additional portions of one or more portions of the feed anaerobic digestate can be added to the cell culture. The one or more portions of the feed anaerobic digestate can be added to the cell culture anytime during the exponential phase. In certain embodiments, the one or more portions of the feed anaerobic digestate can be added to the cell culture at about the mid-exponential phase; about the late-exponential phase; or anytime between the mid-exponential phase to the late-exponential phase. The feed anaerobic digestate can be the same anaerobic digestate present in the cell culture or can be different. The feed anaerobic digestate can be added in one or more portions thereby increasing the carbohydrate concentration in the cell culture during the mid-exponential phase to late-exponential phase to 5 to 40 g/L; 5 to 35 g/L; 5 to 30 g/L; 5 to 25 g/L; 5 to 20 g/L; or 5 to 15 g/L.

Once the carbohydrate concentration in the cell culture has been adjusted by the more portions of the feed anaerobic digestate, the cell culture can be allowed to cultivate for the period of time required for the host cells to metabolize the residual nutrients in the cell culture before harvesting the rhamnolipids. The period of time can vary between 4 to 50 h after addition of the one or more portions of the feed anaerobic digestate until the rhamnolipids are harvested. In certain embodiments, the period of time after addition of the one or more portions of the feed anaerobic digestate is between 4 to 50 h; 16 to 50 h; 28 to 50 h; or 40 to 50 h until the rhamnolipids are harvested. In certain embodiments, the period of time after addition of the one or more portions of the feed anaerobic digestate is 16 to 28 h; 18 to 26 h; or 20 to 24 h until the rhamnolipids are harvested. In certain embodiments, the period of time after addition of the one or more portions of the feed anaerobic digestate is about 22 h until the rhamnolipids are harvested.

The pH of the culture medium can range between 6 to 10. In certain embodiments, the pH is between 6 to 9.5; 7 to 9.5; 7 to 9.0; 7.5 to 9.0; 7.0 to 8.0; or 7.25 to and 7.75.

The C/N ratio of the culture medium can range between 7 to 25; 7 to 22; 10 to 22; 17 to 22; or 20 to 25.

Cultivation of the cell culture can occur at any temperature between 20° C. to 80° C. In certain embodiments, cultivation of the cell culture occurs between 20° C. to 75° C.; 20° C. to 70° C.; 20° C. to 65° C.; 20° C. to 60° C.; 25° C. to 60° C.; 30° C. to 60° C.; 35° C. to 60° C.; 40° C. to 60° C.; 45° C. to 60° C.; or 50° C. to 60° C. The cultivation temperature of the cell culture can be controlled using any conventional method known in the art, such as by circulating cold water.

In the methods described herein the rhamnolipids are recovered. Typically, the rhamnolipids are secreted by the host cell, so that recovering the fermentation/culture medium includes recovering the rhamnolipid.

The methods described herein may optionally include enriching, isolating and/or purifying the rhamnolipid. The term “enriched” means that the rhamnolipids constitute a higher fraction of the mass in the sample of interest than in the sample from which it was taken. Isolating and purifying the rhamnolipids can be accomplished using any conventional technique known in the art. Isolating and/or purifying may for instance include membrane filtration, for example, by buffer exchange or concentration purposes. It may also include filtration or dialysis, which may for instance be directed at the removal of molecules below a certain molecular weight, or a precipitation using organic solvents or ammonium sulfate. Chromatography may for example be carried out in the form of a liquid chromatography such as capillary electrochromatography, HPLC (high performance liquid chromatography) or UPLC (ultrahigh pressure liquid chromatography) or as a gas chromatography. The chromatography technique may be a process of column chromatography, of batch chromatography, of centrifugal chromatography or a method of expanded bed chromatography. Another example of a purification is an electrophoretic technique, such as preparative capillary electrophoresis including isoelectric focusing. Examples of electrophoretic methods are for instance free flow electrophoresis (FFE), polyacrylamide gel electrophoresis (PAGE), capillary zone or capillary gel electrophoresis. An isolation may include the combination of similar methods. Isolating and/or purifying may also include liquid-liquid or liquid-solid extraction of a sample comprising the rhamnolipid; or crystallization of the rhamnolipid.

In certain embodiments, the method for preparing for rhamnolipids is accomplished using a fed-batch culture system and developed in a laboratory bioreactor (Bioengineering, Switzerland) using anaerobic digestate as the fermentation medium (feedstock).

The host cells in the examples herein is Acinetobacter calcoaceticus BU-03, which is stored at −80° C. in 50% glycerol and thawed quickly for use. The production of rhamnolipids can be performed in two pre-culture phases in shake flasks and followed by a production phase in the fermenter. First, single colonies of A. calcoaceticus are obtained on nutrient agar plate upon overnight (15-20 h) incubation at 55° C. For first pre-culture preparation, a single colony from plate is used for inoculation of nutrient broth, which is then incubated at 55° C. for 15-20 h in an orbital shaker incubator rotating at 150 rpm. Overnight grown culture with an optical density (540 nm) of 2.0 is used for inoculation of anaerobic digestate (10% v/v inoculation) in shake flask and incubated for 24 h under the same conditions as used for first pre-culture. This step is necessary to acclimatize the culture to anaerobic digestate before introducing it in the fermenter.

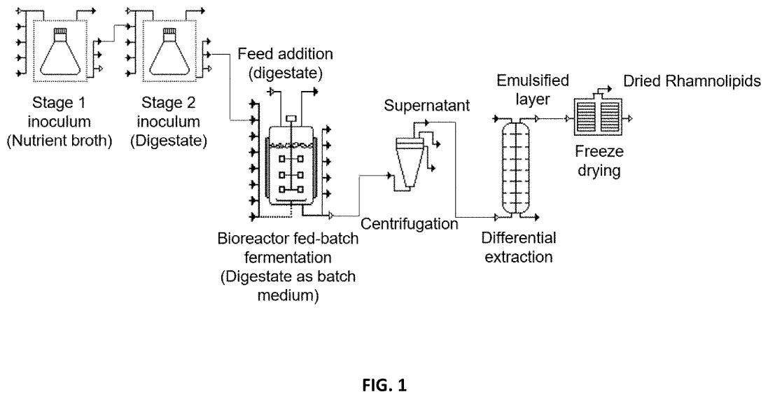

A 2 L fermenter with initial working volume of 1 L can be used for production phase. The anaerobic digestate can be sterilized in-place with the fermenter vessel. Anaerobic digestate is obtained from anaerobic digestion of food waste and has a pH of 8.10, a total solids (TS) content of 2.5% and an electrical conductivity of 7.5 mS/cm. The total carbohydrate concentration is 30 g/L, total organic carbon content of 24.5% and a total nitrogen concentration of 1 g/L. There is no requirement to subject anaerobic digestate to harsh, expensive and complicated pre-treatments steps, such as acid hydrolysis, enzyme treatment, etc. prior to use as fermentation feedstock. The fermentation has three phases. First, it is initiated as a batch cultivation and fermenter is inoculated with 10% (v/v) second pre-culture. Second, at 20 h, when the culture is in late-exponential phase and nutrients start to become limiting, as indicated by dissolved oxygen profile, the feeding of the one or more portions of feed anaerobic digestate is started into the fermenter. Feeding occurs by pulse addition of the one or more portions of feed anaerobic digestate into the fermenter to achieve a final carbohydrates concentration of 10 g/L in the fermentation broth. Third, the cultivation is carried out for another 22 h after feeding to allow the residual nutrient consumption. Agitation in fermenter is achieved by means of two Rushton turbine blade impellers rotating at 400 rpm. Temperature is controlled at 55° C. via circulation through chilled water unit while pH is maintained at 7.5 through addition of 2M NaOH/HCl via peristaltic pumps. The culture broth is harvested at 42 h and rhamnolipids are extracted from supernatant using n-hexane. The concentration of rhamnolipids is measured by standard method of Anthrone assay (Nitschke et al., 2005) of the extracted product. The process flow for rhamnolipids production using anaerobic digestate is shown in FIG. 1.

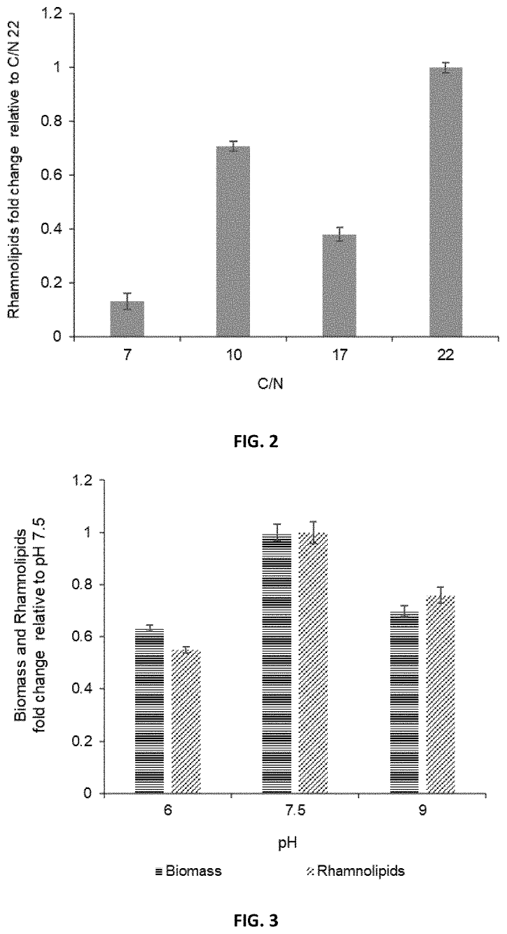

Before the fed-batch fermentation design, the nutrient concentration of anaerobic digestate and cultivation operating parameters which are most optimal for rhamnolipids production are pre-determined by independent batch cultivation experiments. In the first set of experiments, the carbohydrates concentration is varied from 10-30 g/L, total organic carbon content varied from 15-25%, carbon/nitrogen mass ratio (C/N) varied from 7-22, and electrical conductivity, as a measure of salinity, is varied from 7.5-16 mS/cm. Carbohydrates are a type of carbon source in digestate along with other carbon sources such as volatile fatty acids, glycerol etc. High carbohydrates availability in the culture medium supports high cell growth and rhamnolipids production. Consequently, a 1.3-fold increase in biomass growth is seen with a 2-fold increase in carbohydrates concentration in the digestate. However, rhamnolipids being secondary metabolites, their production is not favored unless there is a some nutrient limitation. This implies that only high carbohydrates is not sufficient and the nutrients are diverted to build biomass rather than to produce rhamnolipids if nitrogen is also highly available. Therefore, in addition to carbohydrates concentration, the C/N plays an important role in determining the rhamnolipids production and a high C/N ratio i.e. a high carbon and a low nitrogen content is ideal for rhamnolipids (i.e. a high C/N ratio of anaerobic digestate). A 7-fold higher rhamnolipids concentration is obtained at the highest C/N level tested in these experiments as compared to that achieved on the lowest C/N (7) tested (FIG. 2). Additionally, high salinity of the anaerobic digestate can inhibit rhamnolipids production, e.g., a 2.5-fold lower rhamnolipids is obtained with a 2-fold increase in salinity of digestate when used as the culture medium.

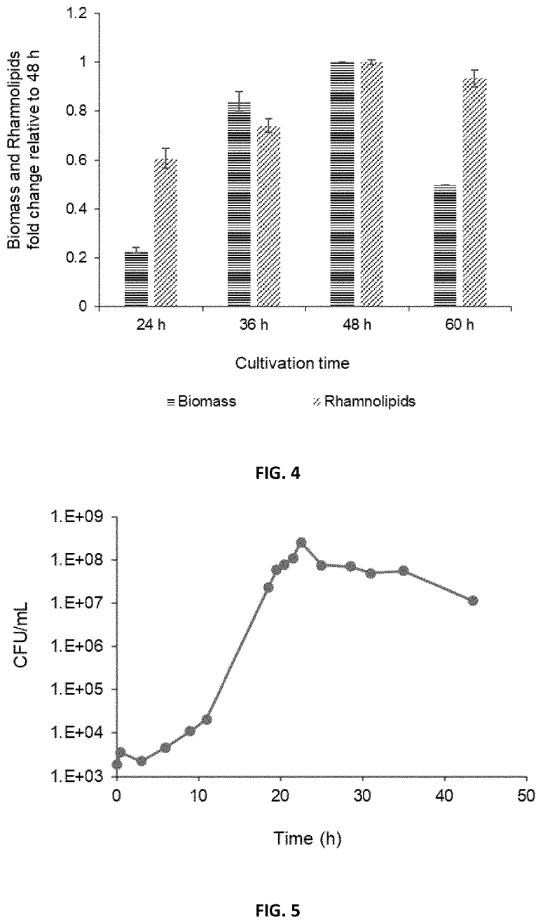

The operating conditions of pH, temperature and cultivation time are varied in the second set of experiments. pH is one of the most important environmental factors which influences growth and metabolite production predominantly due to its effect on enzyme activity. To investigate the effect of pH variation of anaerobic digestate, pH is adjusted to reach a final pH value of 6.0, 7.5 and 9.0 and then the anaerobic digestate is used as the production medium. pH 7.5 results in a rhamnolipids enhancement by 31.2% as compared to pH 9.0. On the other hand, a 45.2% decrease is observed at pH 6.0 versus pH 7.5 (FIG. 3). Similar to pH, temperature is required for optimal activity of enzymes involved in cellular metabolic pathways. The effect of two temperatures, i.e., 37° C. (mesophilic) and 55° C. (thermophilic) on rhamnolipids production using anaerobic digestate (at pH 7.5) is investigated. While the culture exhibits biomass growth at both temperatures, it is reduced by 61.2% when grown on 37° C. Similarly, a 73% reduction is observed for rhamnolipids production at a lower cultivation temperature. Therefore, a high temperature is more suitable to solubilize the macromolecules in anaerobic digestate and make them available for consumption by the rhamnolipids producer during fermentation. Production of rhamnolipids is dependent on biomass accumulation. Therefore, it is pertinent to determine the required cultivation time necessary to allow high biomass and associated high rhamnolipids production. Obtaining high rhamnolipid concentration in minimum time would be desirable to achieve a high overall productivity of the bioprocess. Thus, the influence of cultivation time on rhamnolipid production using anaerobic digestate (at pH 7.5, temperature 55° C.) is investigated and the cultivation time is varied as 24 h, 36 h, 48 h and 60 h. A gradual increase in culture growth is seen from 24 h until 48 h. The increase in culture growth is drastic from 24 h to 36 h which is understandable since additional 12 h (from 24 h) allows reasonable time for the culture to grow. Since rhamnolipids production occurs from mid-exponential phase until stationary phase, the accumulated biomass until 36 h could produce higher rhamnolipids when the cultivation time is further increased to 48 h. After this time, the culture growth deteriorates at 60 h while no significant change occurs in rhamnolipids synthesis (FIG. 4). This clearly implies that extending the production time does not help due to the culture decline and therefore 40-50 h is a suitable cultivation time for rhamnolipids production on anaerobic digestate. Finally, the above conditions were used for design of the fed-batch fermentation process.

The fed-batch fermentation is performed using food waste anaerobic digestate both as initial batch medium and as feed during the feeding phase. The result shows that anaerobic digestate supports a very good biomass growth from the beginning of cultivation (FIG. 5). The biomass concentration as estimated by colony forming units (CFU) per mL demonstrates that a CFU/mL of 5.8×10⁷ can be reached during the batch cultivation on anaerobic digestate. Upon supply of additional nutrients by feeding of anaerobic digestate at 20 h, the biomass continues to increase further and reaches a peak concentration of 2.57×10⁸. The nutrients in the culture medium should meet the basic requirements for cell biomass growth and metabolite production by providing the adequate supply of energy for biosynthesis and cell maintenance. The carbon and nitrogen sources in the medium are most important for biosynthesis and energy generation and for initiating the biosynthesis of precursors for metabolite production. As can be seen from biomass growth profile in FIG. 5, the anaerobic digestate supports a high biomass accumulation which indicates that it contains all the required nutrient sources. This result is significant since there is no requirement of addition of expensive salts, vitamins, growth factors, mineral sources, etc. to anaerobic digestate to support biomass growth.

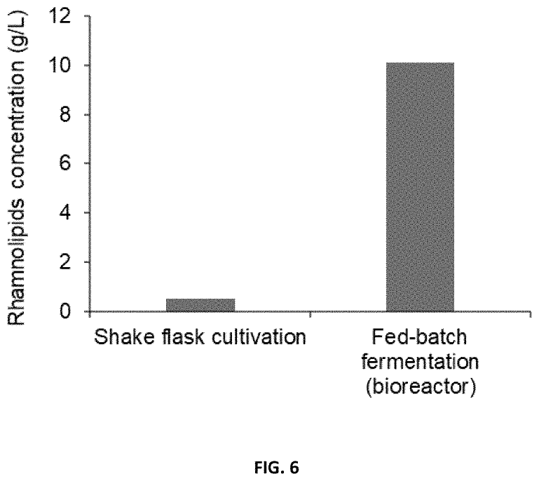

Regarding the rhamnolipids production, the accumulated biomass can produce a rhamnolipids concentration of up to 10 g/L using this fed-batch fermentation method in a short period of only 42 h. In comparison, the shake flask method can only produce ˜0.5 g/L rhamnolipids concentration (FIG. 6). The chemical characterisation of rhamnolipids is performed and the predominant rhamnolipid congeners are Rha-C10-C10 and Rha-Rha-C10-C10.

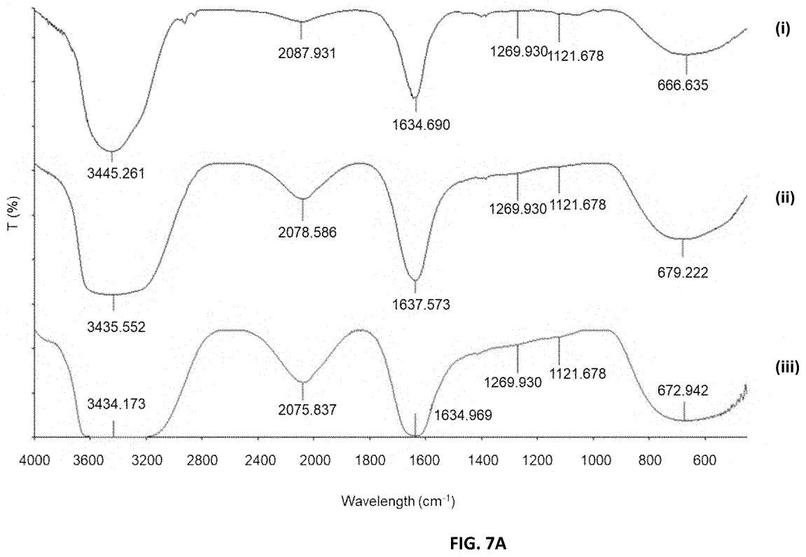

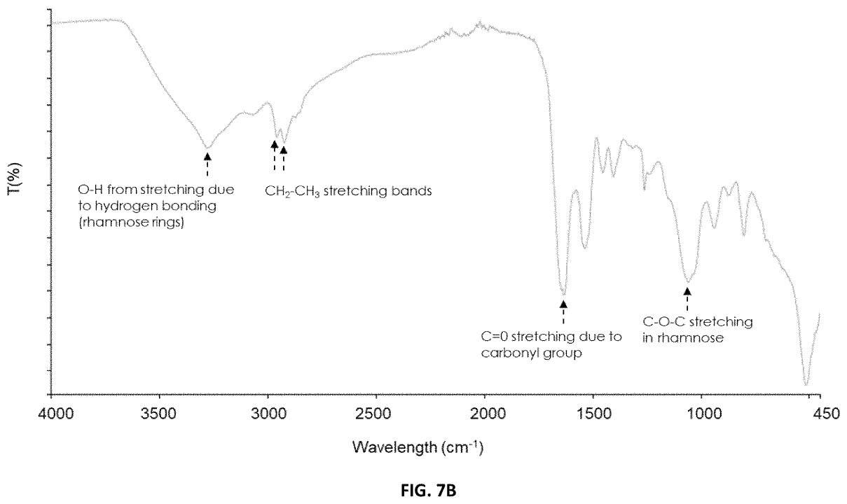

The characterization of extracted rhamnolipids is performed by Fourier-Transform Infrared (FTIR) spectroscopy (FIGS. 7A and 7B). Rhamnolipids standard, anaerobic digestate-derived rhamnolipids and synthetic medium-derived rhamnolipids are analysed and all three had similar absorption bands. Rhamnolipids R90 obtained from Sigma (USA) is used as the standard. The bands are consistent with previous reports (Guo et al., 2009; Kiefer et al., 2017). The important absorption bands corresponding to rhamnolipids are seen. The broad band at 3434 cm⁻¹ shows the presence of O—H bond. The peak at 1269 cm⁻¹ corresponds to C—O bond. The spectrum of rhamnolipids peaks at 3434-3580 cm⁻¹ (O—H from stretching due to hydrogen bonding), 1637 cm⁻¹ (C═O stretching due to ester functional group), and 1121 cm⁻¹ (C—O—C stretching in rhamnose). The characteristic adsorption bands demonstrate that anaerobic digestate-derived rhamnolipids hold chemical structure identical to those of standard rhamnolipids, and those reported in literature, thus reinforcing the fact that anaerobic digestate is a suitable feedstock for rhamnolipids production.

The chemical characterization is performed by direct infusion into MS (Déziel et al., 2000). The injection is performed using a 40% acetonitrile/water solution containing 4 mM of ammonium acetate at a flow rate of 40 μL/min. The first injection is performed in full scan mode with a mass range of 400-750 Da. Quantification is performed with the pseudomolecular ions. For isomeric rhamnolipids, the relative proportion of the two isomers is obtained with a second injection with a MS/MS method using multiple reaction monitoring (MRM) mode.

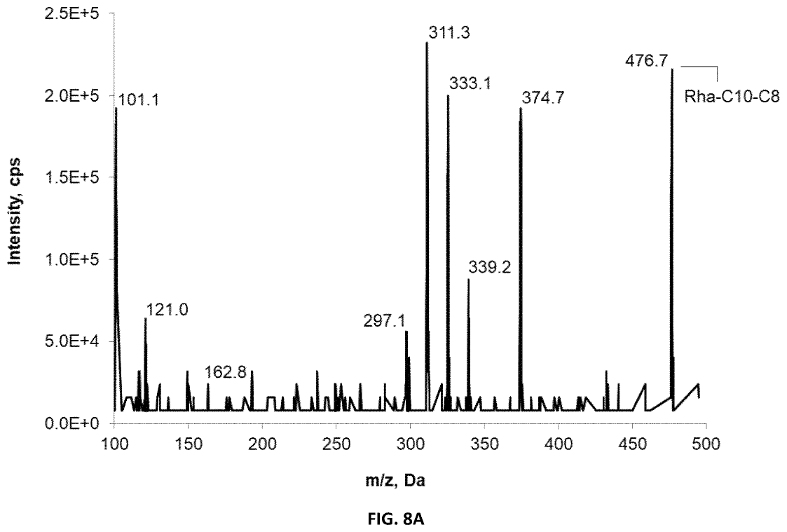

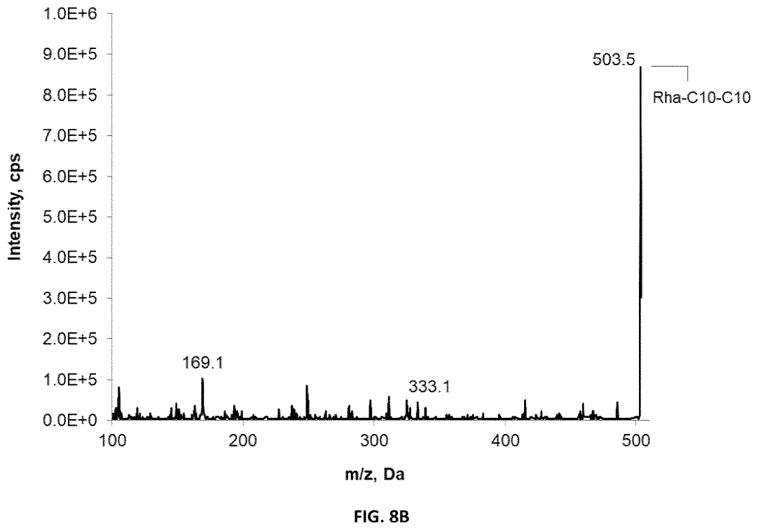

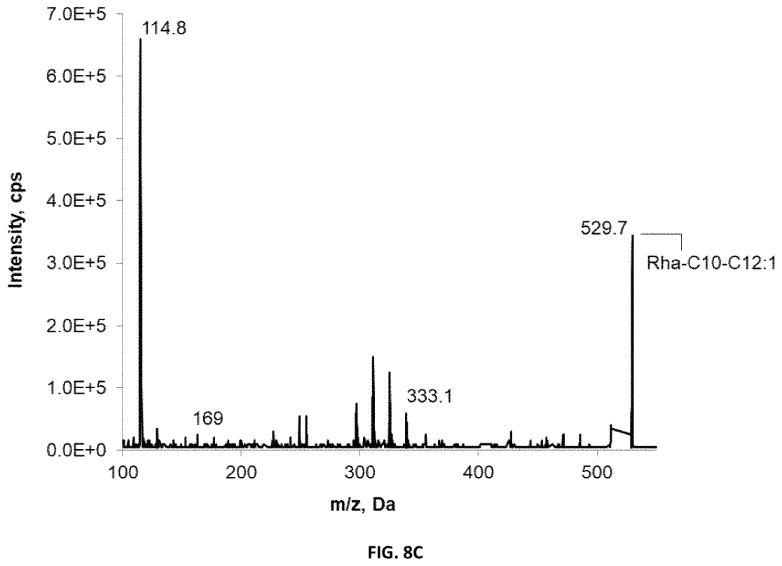

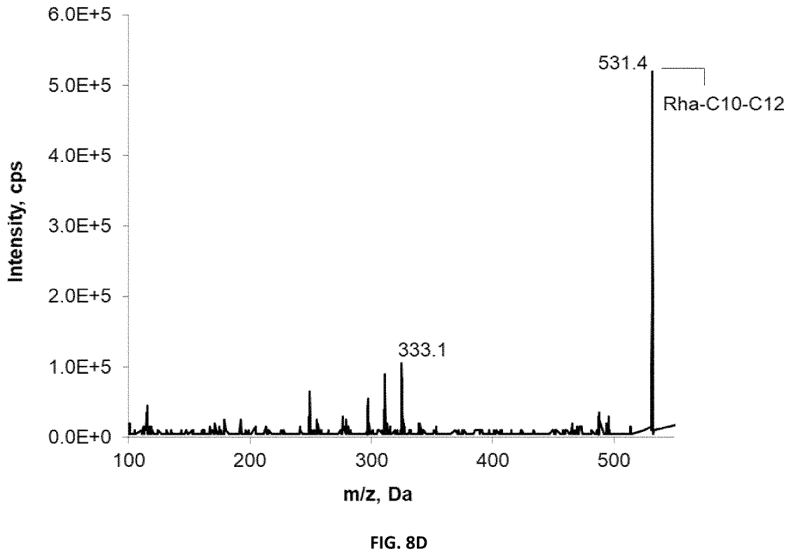

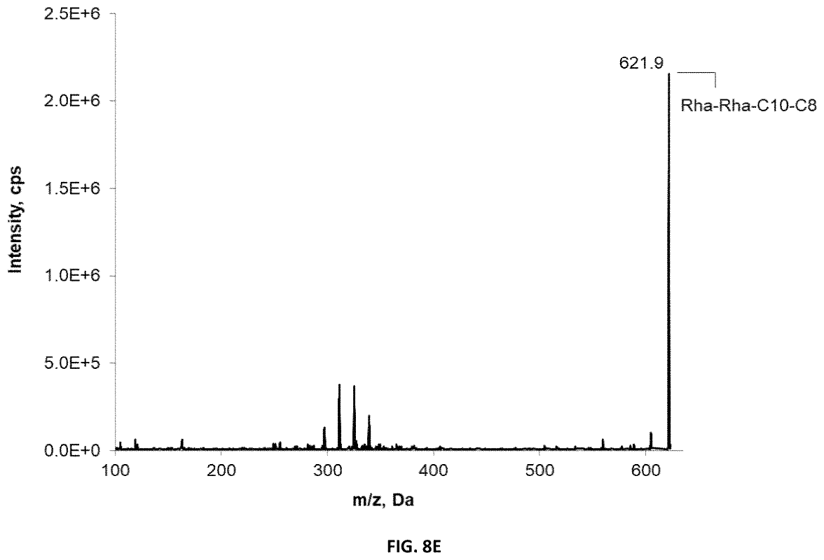

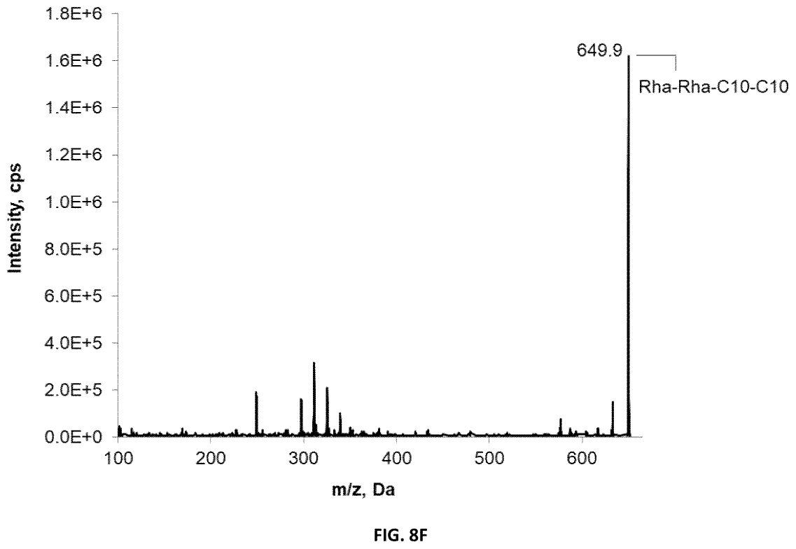

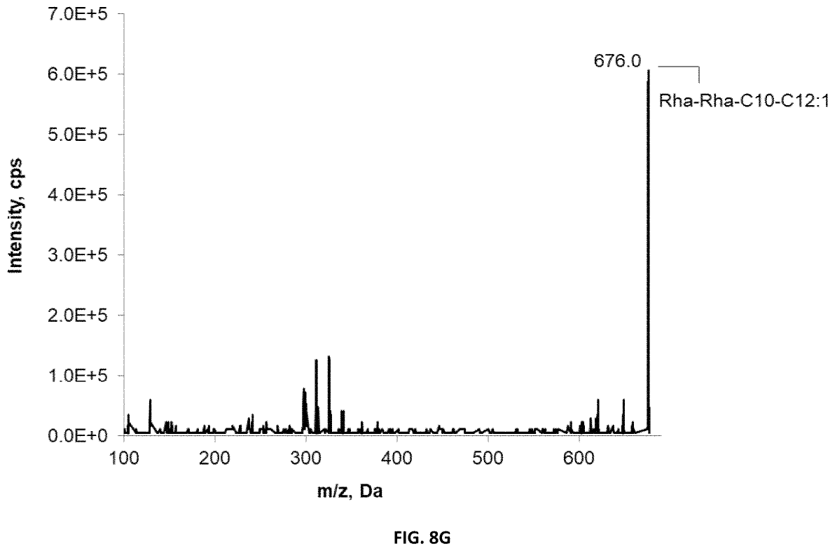

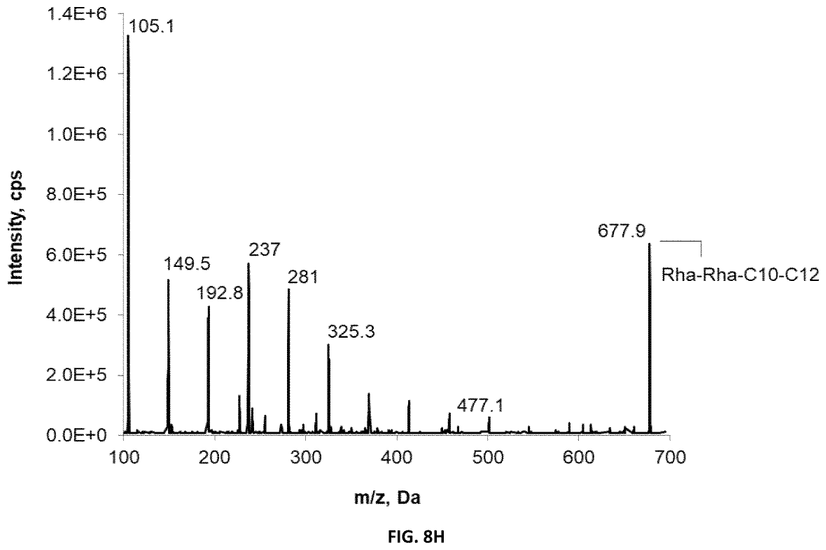

The results for direct infusion in MS are shown in FIGS. 8A-8H and the relative abundance of congeners is described in Table 1. As shown in the table, the predominant rhamnolipid congeners are mono-rhamnolipids Rha-C10-C10 and di-rhamnolipids Rha-Rha-C10-C10 and minor fractions of other molecules are present. These results are consistent with the classical report by Rooney et al. (2009) who performed a detailed characterization of rhamnolipids produced by Acinetobacter calcoaceticus NRRL B-59190 and Acinetobacter calcoaceticus NRRL B-59191 and found that the predominant molecules were mono- and di-rhamno-C10-C10.

TABLE 1
Rhamnolipid congeners synthesized using anaerobic
digestate in bioreactor cultivation and the percentage
relative abundance of each congener.

| | | | MRM 2 | Relative |
|---|---|---|---|---|
| | | MRM 1 | (confir- | Abundance |
| Compounds | m/z | (analytical) | matory) | (%) |
| Rha-C10-C10 | 503.00 | 503.3/169.0 | 503.3/333.0 | 34.44 |
| Rha-Rha-C10-C10 | 649.00 | 649.4/169 | 649.4/479.2 | 39.74 |
| Rha-C10-C8 | 476 | 476.4/162.8 | 476.4/332.9 | 2.445 |
| Rha-C10-C12:1 | 529.3 | 529.3/169.0 | 529.3/333.0 | 2.083 |
| Rha-Rha-C10-C8 | 621.5 | 621.5/169.1 | 621.5/479.4 | 3.27 |
| Rha-Rha-C10-C12 | 677.5 | 677.5/169.0 | 677.5/479.2 | 6.55 |
| Rha-Rha-C10-C12:1 | 675.5 | 675.5/169.1 | 675.5/479.2 | 6.54 |
| Rha-C10-C12 | 531.1 | 531.3/168.9 | 531.3/333.1 | 4.918 |

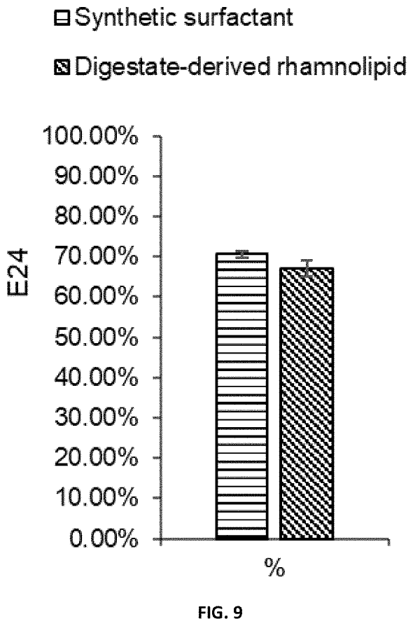

The emulsification index E24 assay for extracted rhamnolipids is performed using the protocol as given by Dobler et al. (2017). Briefly, equal amount of rhamnolipid solution (1 g/L) and n-hexadecane (reference for fuel mixtures/cetane number 100) are mixed using a vortex mixer at maximum level for 5 min and subsequently allowed to stand for 24 h. E24 index is estimated by the ratio between the emulsion volume and total volume. Tween 80, a synthetic surfactant, taken at same concentration as rhamnolipids is used for comparison. The result is shown in FIG. 9 and it shows that the E24 index of anaerobic digestate-produced rhamnolipids was 67%. This emulsification activity was only slightly lower than that of synthetic surfactant which showed an E24 index of 70.6%. This further indicates that anaerobic digestate-derived rhamnolipids are comparable to synthetic surfactants and exhibit a high surfactant activity, thereby showing a great commercial value.

The present disclosure is not to be limited in scope by any of the specific embodiments described herein. The specific embodiments described herein are presented for exemplification only.

### INDUSTRIAL APPLICABILITY

The present disclosures relates to a to a bio-based, cost-effective and sustainable method and system for production of rhamnolipids using digestate. In particular, the present disclosure provides economic incentives for AD plants to produce a high-value commodity by utilizing their waste and provides an environment-friendly and efficient waste management system for digestate.

What is claimed is:

1. A method for producing rhamnolipids, the method comprising:
providing a culture medium comprising an anaerobic digestate and a host cell which produces rhamnolipids; cultivating the host cell under conditions that the host cell produces the rhamnolipids;
recovering the rhamnolipids; and
optionally isolating the rhamnolipids.

2. The method of claim 1, wherein the anaerobic digestate is untreated.

3. The method of claim 1, wherein the anaerobic digestate is substantially the only carbon source and nitrogen source in the culture medium.

4. The method of claim 1, wherein the host cell is Acinetobacter calcoaceticus.

5. The method of claim 1, wherein the culture medium has a carbohydrates concentration of 10 to 30 g/L and a carbon to nitrogen ratio mass ratio between 7 to 22.

6. The method of claim 1, wherein the host cell is cultivated at a temperature of 20° C. to 60° C.

7. The method of claim 1, wherein the culture medium has a pH of 6 to 9.

8. The method of claim 1, further comprising adding one or more additional portions of a feed anaerobic digestate into the culture medium during about the mid-exponential growth phase of the host cell to about the late-exponential growth phase of the host cell thereby forming a fermentation culture having a carbohydrate concentration in the fermentation culture between 10 to 30 g/L and a carbon to nitrogen ratio mass ratio of 7 to 22.

9. A method for producing rhamnolipids, the method comprising:
providing a culture medium comprising an anaerobic digestate and Acinetobacter calcoaceticus under conditions that the Acinetobacter calcoaceticus produces the rhamnolipids, wherein the conditions comprise cultivating the Acinetobacter calcoaceticus until about mid-exponential growth phase of the Acinetobacter calcoaceticus to the late-exponential growth phase of the Acinetobacter calcoaceticus;
adding one or more additional portions of a feed anaerobic digestate into the culture medium thereby forming a fermentation culture having a carbohydrate concentration in the fermentation culture between 10 to 30 g/L and a carbon to nitrogen ratio mass ratio of 7 to 22;
recovering the rhamnolipids; and optionally isolating the rhamnolipids, wherein the anaerobic digestate and the one or more portions of the feed anaerobic digestate are substantially the only carbon source and nitrogen source in the culture medium.

10. The method of claim 9, wherein the anaerobic digestate and the one or more portions of the feed anaerobic digestate are untreated.

11. The method of claim 10, wherein the anaerobic digestate and the one or more portions of the feed anaerobic digestate are prepared by anaerobic digestion of food waste.

12. The method of claim 11, wherein the anaerobic digestate and the one or more portions of the feed anaerobic digestate have a pH between 6 to 9 an electrical conductivity between 7.5 to 16 mS/cm.

13. The method of claim 9, wherein the culture medium has a pH between 6 and 9 and a temperature of 20° C. to 60° C.

14. The method of claim 9, further comprising: providing a first pre-culture medium comprising Acinetobacter calcoaceticus; combining the first pre-culture medium and a first portion of the anaerobic digestate thereby forming a second pre-culture medium; and combining the second pre-culture medium with a second portion of the anaerobic digestate thereby forming the culture medium.

15. The method of claim 9, further comprising: cultivating the fermentation culture between 4 to 50 hours (h) before the step of recovering the rhamnolipids.

16. The method of claim 9, wherein the method comprises:
providing a culture medium comprising an anaerobic digestate and Acinetobacter calcoaceticus under conditions that the Acinetobacter calcoaceticus produces the rhamnolipids, wherein the conditions comprise cultivating the Acinetobacter calcoaceticus until about late-exponential growth phase of the Acinetobacter calcoaceticus;
adding one or more additional portions of a feed anaerobic digestate into the culture medium thereby forming a fermentation culture having a carbohydrate concentration in the fermentation culture between 10 to 30 g/L and a carbon to nitrogen ratio mass ratio between 10 to 22;
cultivating the fermentation culture between 20 to 30 h, wherein the fermentation culture has a pH between 7.5 to 9 and a temperature between 50° C. and 60° C.;
harvesting the rhamnolipids; and optionally isolating the rhamnolipids, wherein the anaerobic digestate and the one or more portions of the feed anaerobic digestate are substantially the only carbon source and nitrogen source in the culture medium and the fermentation culture, and wherein the anaerobic digestate and the one or more portions of the feed anaerobic digestate are untreated; have a pH between 7.5 to 9; and an electrical conductivity between 7.5 to 16 mS/cm.

17. The method of claim 16, further comprising providing a first pre-culture medium comprising Acinetobacter calcoaceticus;
combining the first pre-culture medium and a first portion of the anaerobic digestate thereby forming a second pre-culture medium; and
combining the second pre-culture medium with a second portion of the anaerobic digestate thereby forming a culture medium comprising anaerobic digestate and Acinetobacter calcoaceticus.

18. The method of claim 16, wherein the anaerobic digestate and the one or more portions of the feed anaerobic digestate are prepared by anaerobic digestion of food waste.

19. The method of claim 16, wherein the fermentation culture produces rhamnolipids at a concentration of 8 to 12 g/L.

20. The method of claim 16, wherein the rhamnolipids are isolated by liquid-liquid extraction of the fermentation culture with an organic solvent.
